SEQUENCE LISTING (10 sequences)

SEQ ID NO: 1 - DNA, 359 bp, Mus musculus
  CDS (1)...(359): 16E10 heavy chain v region
caggttcaactaaaggagtcaggacctggcctggtggcgccctcacagagcctgtccatcacatgcaccgtctcagggttctcattaaccggctatggtgtaaactgggttcgccagcctccaggaaagggtctggagtggctgggaatgatttgggttgatggaggcacagactataattcagctctcaaatccagactgagcatcagcaaggacaactccaagagccaagttttcttaaaaatgaacagtctgcaaactgatgacacagccaggtactactgtgccagaggagggagttcattactggggtttgcttactggggccaagggactctggtcactgtctctgcagccaa

SEQ ID NO: 2 - protein, 119 aa, Mus musculus
QVQLKESGPGLVAPSQSLSITCTVSGFSLTGYGVNWVRQPPGKGLEWLGMIWVDGGTDYNSALKSRLSISKDNSKSQVFLKMNSLQTDDTARYYCARGGSSLLGFAYWGQGTLVTVSAA

SEQ ID NO: 3 - DNA, 324 bp, Mus musculus
  CDS (1)...(324): 16E10 light chain v region
gatatacagatgactcagactacatcctccctgtctgcctctctgggagacagagtcaccatcagttgcagggcaagtcaggacattagcaattatttaaactggtatcaacagaaaccagatggaactgttaaactcctgatctactacacatcaagattacactcaggagtcccatcaaggttcagtggcagtgggtctggaacagattattctctcaccattagcaacctggagcaagaagatattgccacttacttttgccaacagggtaatacgcttcctcggacgttcggtggaggcaccaagctggaaatcaaacgg

SEQ ID NO: 4 - protein, 108 aa, Mus musculus
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPRTFGGGTKLEIKR

SEQ ID NO: 5 - protein, 5 aa, Mus musculus
  SITE (1)...(5): heavy chain CDR 1
GYGVN

SEQ ID NO: 6 - protein, 16 aa, Mus musculus
  SITE (1)...(16): heavy chain CDR 2
MIWVDGGTDYNSALKS

SEQ ID NO: 7 - protein, 10 aa, Mus musculus
  SITE (1)...(10): heavy chain CDR 3
GGSSLLGFAY

SEQ ID NO: 8 - protein, 11 aa, Mus musculus
  SITE (1)...(11): light chain CDR 1
RASQDISNYLN

SEQ ID NO: 9 - protein, 7 aa, Mus musculus
  SITE (1)...(7): light chain CDR 2
YTSRLHS

SEQ ID NO: 10 - protein, 9 aa, Mus musculus
  SITE (1)...(9): light chain CDR 3
QQGNTLPRT

(19) United States
(12) Patent Application Publication
(10) Pub. No.: US 2002/0127227 A1
(43) Pub. Date: Sep. 12, 2002

(54) RHAMM antagonist antibodies

(75) Inventors: Stephen D. Holmes, Great Chishill (GB); Julie A. Abrahamson, Harlow (GB); Jeffrey R. Jackson, Schwenksville, PA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 10/140,555

(22) Filed: May 7, 2002

Related U.S. Application Data
(63) Continuation of application No. 09/443,790, filed on Nov. 19, 1999.
(60) Provisional application No. 60/109,041, filed on Nov. 19, 1998.
(60) Provisional application No. 60/143,692, filed on Jul. 14, 1999.

(51) Int. Cl. A61K 39/395; C07H 21/04; C07K 16/30

(52) U.S. Cl. 424/143.1; 530/388.22; 536/23.53

(57) ABSTRACT

The present invention provides antagonist monoclonal antibodies to human RHAMM (receptor for hyaluronic acid mediated notility) and to the use of these monoclonal antibodies as therapeutics for the treatment of proliferative diseases.

### RELATED APPLICATIONS

This application claims the benefit of U.S. Provisional Application No. 60/109,041, filed Nov. 19, 1998 and U.S. Provisional Application No. 60/143,692, filed Jul. 14, 1999.

### FILED OF THE INVENTION

This invention relates to antagonist monoclonal antibodies (mAb) that bind to the RHAMM (receptor for hyaluronic acid mediated motility) receptor and to the use of such antibodies for therapeutic purposes.

### BACKGROUND OF THE INVENTION

Hyaluronic acid (HA) is a high molecular weight (avg. is several million daltons) glycosaminoglycan found in the extracellular matrix as well as interstitial and synovial fluids. It plays a normal role in water and plasma protein homeostasis and cell migration during development. It also plays an important role in tumorigenesis and inflammation, stimulating both cellular proliferation and migration. RHAMM is a Receptor for Hyaluronic Acid Mediated Motility. Of the known HA receptors, which also include CD44 and ICAM, RHAMM has been demonstrated to be required for growth factor signaling through ras.

Murine RHAMM mediates protein tyrosine phosphorylation and focal adhesion turnover in response to HA (Hall et al, 1994, J Cell Biol, 126:575). In addition, RHAMM is required for H-ras transformation, and normal fibroblasts overexpressing RHAMM are capable of forming tumors and spontaneous metastases (Hall et al, 1995, Cell, 82:19). This study also demonstrated that expression of a dominant negative RHAMM could suppress tumor formation by ras-transformed fibroblasts. Most recently, RHAMM has been shown to be required for ERK (p42 MAP kinase) activation by PDGF and ras (Zhang et al, 1998, J Biol Chem, 273:11342). High levels of HA production are associated with tumor growth and other proliferative diseases such as rheumatoid arthritis. RHAMM appears to be responsible for cellular proliferation and migration in response to HA. Antibodies to murine RHAMM were able to functionally block migration in response to HA (Hall et al, 1994, J Cell Biol, 126:575) and growth factor signaling through the ras pathway (Zhang et al, 1998, J Biol Chem, 273:11342).

Thus, a monoclonal antibody against human RHAMM would be useful as a therapeutic agent to treat ras-dependent proliferation; and particularly, antagonist monoclonal antibodies would be beneficial in treatment of proliferative disorders including leukemias, solid tumor cancers and metastases such as lymphomas, soft tissue, brain, esophageal, stomach, pancreatic, liver, lung, bladder, bone, prostate, ovarian, cervical, uterine, skin, breast, testicular, kidney, head and neck, and colon cancers; chronic inflammatory proliferative diseases such as psoriasis, inflammatory bowel disease and rheumatoid arthritis; proliferative cardiovascular diseases such as restenosis; proliferative ocular disorders such as diabetic retinopathy; and benign hyperproliferative diseases such as hemangiomas.

### SUMMARY OF THE INVENTION

One aspect of the present invention includes a monoclonal antibody against human RHAMM (receptor for hyaluronic acid mediated motility) having the identifying characteristics of monoclonal antibody 10C5, 16E10 or 3E6. Another aspect to the present invention includes a method for treating or preventing proliferative disease states in a mammal comprising administering to a subject in need thereof an effective dose of a RHAMM receptor antagonist antibody having the identifying characteristics of monoclonal antibody 10C5, 16E10 or 3E6. Another aspect of the present invention includes a pharmaceutical composition comprising a monoclonal antibody against human RHAMM having the identifying characteristics of monoclonal antibody 10C5, 16E10 or 3E6.

### BRIEF DESCRIPTION OF THE DRAWING

FIG. 1 demonstrates the ability of RHAMM monoclonal antibodies 10C5, 16E10 and 3E6 to inhibit the binding of Hyaluronic acid to RHAMM in a microwell binding assay.

### DETAILED DESCRIPTION OF THE INVENTION

All publications, including but not limited to patents and patent applications, cited in this specification are herein incorporated by reference as though fully set forth.

As used herein, the term “proliferative disease state” refers to any disease in which cellular proliferation, either malignant or benign, contributes to the pathology of the condition. Such unwanted proliferation is the hallmark of cancer and many chronic inflammatory diseases, thus examples of “proliferative disease states” include leukemias, solid tumor cancers and metastases such as lymphomas, soft tissue, brain, esophageal, stomach, pancreatic, liver, lung, bladder, bone, prostate, ovarian, cervical, uterine, skin, breast, testicular, kidney, head and neck, and colon cancers; chronic inflammatory proliferative diseases such as psoriasis, inflammatory bowel disease and rheumatoid arthritis; proliferative cardiovascular diseases such as restenosis; proliferative ocular disorders such as diabetic retinopathy; and benign hyperproliferative diseases such as hemangiomas.

As used herein, the term “antagonist activity” refers to the activity of an antibody against the RHAMM receptor to inhibit binding of hyaluronic acid.

As used herein, the term “treating” and “preventing” means prophylactic or therapeutic therapy.

“Monoclonal Antibodies” refers to immunoglobulins which can be prepared by conventional hybridoma techniques, phage display combinatorial libraries, immunoglobulin chain shuffling and humanization techniques. Also included are fully human monoclonal antibodies. As used herein, “antibody” also includes “altered antibody” which refers to a protein encoded by an altered immunoglobulin coding region, which may be obtained by expression in a selected host cell. Such altered antibodies are engineered antibodies (e.g., chimeric or humanized antibodies) or antibody fragments lacking all or part of an immunoglobulin constant region, e.g., Fv, Fab, Fab′ or F(ab′)₂ and the like.

“Altered immunoglobulin coding region” refers to a nucleic acid sequence encoding an altered antibody of the invention. When the altered antibody is a complementarily determining region-grafted (CDR-grafted) or humanized antibody, the sequences that encode the CDRs from a non-human immunoglobulin are inserted into a first immunoglobulin partner comprising human variable framework sequences. Optionally, the first immunoglobulin partner is operatively linked to a second immunoglobulin partner.

“First immunoglobulin partner” refers to a nucleic acid sequence encoding a human framework or human immunoglobulin variable region in which the native (or naturally-occurring) CDR-encoding regions are replaced by the CDR-encoding regions of a donor antibody. The human variable region can be an immunoglobulin heavy chain, a light chain (or both chains), an analog or functional fragments thereof. Such CDR regions, located within the variable region of antibodies (immunoglobulins) can be determined by known methods in the art. For example Kabat et al. in “Sequences of Proteins of Immunological Interest”, 4th Ed., U.S. Department of Health and Human Services, National Institutes of Health (1987) disclose rules for locating CDRs. In addition, computer programs are known which are useful for identifying CDR regions/structures.

“Second immunoglobulin partner” refers to another nucleotide sequence encoding a protein or peptide to which the first immunoglobulin partner is fused in frame or by means of an optional conventional linker sequence (i.e., operatively linked). Preferably, it is an immunoglobulin gene. The second immunoglobulin partner may include a nucleic acid sequence encoding the entire constant region for the same (i.e., homologous, where the first and second altered antibodies are derived from the same source) or an additional (i.e., heterologous) antibody of interest. It may be an immunoglobulin heavy chain or light chain (or both chains as part of a single polypeptide). The second immunoglobulin partner is not limited to a particular immunoglobulin class or isotype. In addition, the second immunoglobulin partner may comprise part of an immunoglobulin constant region, such as found in a Fab, or F(ab)₂ (i.e., a discrete part of an appropriate human constant region or framework region). Such second immunoglobulin partner may also comprise a sequence encoding an integral membrane protein exposed on the outer surface of a host cell, e.g., as part of a phage display library, or a sequence encoding a protein for analytical or diagnostic detection, e.g., horseradish peroxidase, β-galactosidase, etc.

The terms Fv, Fc, Fd, Fab, Fab′ or F(ab′)₂ are used with their standard meanings. See, e.g., Harlow et al. in “Antibodies A Laboratory Manual”, Cold Spring Harbor Laboratory, (1988).

As used herein, an “engineered antibody” describes a type of altered antibody, i.e., a full-length synthetic antibody (e.g., a chimeric or humanized antibody as opposed to an antibody fragment) in which a portion of the light and/or heavy chain variable domains of a selected acceptor antibody are replaced by analogous parts from one or more donor antibodies which have specificity for the selected epitope. For example, such molecules may include antibodies characterized by a humanized heavy chain associated with an unmodified light chain (or chimeric light chain), or vice versa. Engineered antibodies may also be characterized by alteration of the nucleic acid sequences encoding the acceptor antibody light and/or heavy variable domain framework regions in order to retain donor antibody binding specificity. These antibodies can comprise replacement of one or more CDRs (preferably all) from the acceptor antibody with CDRs from a donor antibody described herein.

A “chimeric antibody” refers to a type of engineered antibody which contains a naturally-occurring variable region (light chain and heavy chains) derived from a donor antibody in association with light and heavy chain constant regions derived from an acceptor antibody.

A “humanized antibody” refers to a type of engineered antibody having its CDRs derived from a non-human donor immunoglobulin, the remaining immunoglobulin-derived parts of the molecule being derived from one or more human immunoglobulins. In addition, framework support residues may be altered to preserve binding affinity. See, e.g., Queen et al., Proc. Natl Acad Sci USA, 86, 10029-10032 (1989), Hodgson et al., Bio/Technology, 9, 421 (1991). Furthermore, as described herein, additional residues may be altered to preserve the antagonist activity of the donor antibody.

The term “donor antibody” refers to a monoclonal or recombinant antibody which contributes the nucleic acid sequences of its variable regions, CDRs or other functional fragments or analogs thereof to a first immunoglobulin partner, so as to provide the altered immunoglobulin coding region and resulting expressed altered antibody with the antigenic specificity and neutralizing activity characteristic of the donor antibody.

The term “acceptor antibody” refers to monoclonal or recombinant antibodies heterologous to the donor antibody, which contributes all, or a portion, of the nucleic acid sequences encoding its heavy and/or light chain framework regions and/or its heavy and/or light chain constant regions or V region subfamily consensus sequences to the first immunoglobulin partner. Preferably, a human antibody is the acceptor antibody.

“CDRs” are defined as the complementarily determining region amino acid sequences of an antibody which are the hypervariable regions of immunoglobulin heavy and light chains. See, e.g., Kabat et al., Sequences of Proteins of Immunological Interest, 4th Ed., U.S. Department of Health and Human Services, National Institutes of Health (1987). There are three heavy chain and three light chain CDRs or CDR regions in the variable portion of an immunoglobulin. Thus, “CDRs” as used herein refers to all three heavy chain CDRs, or all three light chain CDRs or both all heavy and all light chain CDRs, if appropriate.

CDRs provide the majority of contact residues for the binding of the antibody to the antigen or epitope. CDRs of interest in this invention are derived from donor antibody variable heavy and light chain sequences, and include analogs of the naturally occurring CDRs, which analogs share or retain the same antigen binding specificity and/or antagonist ability as the donor antibody from which they were derived, yet may exhibit increased affinity for the antigen. An exemplary process for obtaining analogs is affinity maturation by means of phage display technology as reviewed by Hoogenboom, Trends in Biotechnology 15, 62-70 (1997); Barbas et al., Trends in Biotechnology 14, 230-234 (1996); and Winter et al., Ann. Rev. Immunol. 12, 433-455 (1994) and described by Irving et al., Immunotechnology 2, 127-143 (1996).

A “functional fragment” is a partial heavy or light chain variable sequence (e.g., minor deletions at the amino or carboxyl terminus of the immunoglobulin variable region) which retains the same antigen binding specificity and/or antagonist ability as the antibody from which the fragment was derived.

An “analog” is an amino acid sequence modified by at least one amino acid, wherein said modification can be chemical or a substitution or a rearrangement of a few amino acids (i.e., no more than 10) and corresponding nucleic acid sequences, which modification permits the amino acid sequence to retain the biological characteristics, e.g., antigen specificity and high affinity, of the unmodified sequence. Exemplary nucleic acid analogs include silent mutations which can be constructed, via substitutions, to create certain endonuclease restriction sites within or surrounding CDR-encoding regions.

Analogs may also arise as allelic variations. An “allelic variation or modification” is an alteration in the nucleic acid sequence encoding the amino acid or peptide sequences of the invention. Such variations or modifications may be due to degeneracy in the genetic code or may be deliberately engineered to provide desired characteristics. These variations or modifications may or may not result in alterations in any encoded amino acid sequence.

The term “effector agents” refers to non-protein carrier molecules to which the altered antibodies, and/or natural or synthetic light or heavy chains of the donor antibody or other fragments of the donor antibody may be associated by conventional means. Such non-protein carriers can include conventional carriers used in the diagnostic field, e.g., polystyrene or other plastic beads, polysaccharides, e.g., as used in the BIAcore (Pharmacia) system, or other non-protein substances useful in the medical field and safe for administration to humans and animals. Other effector agents may include a macrocycle, for chelating a heavy metal atom or radioisotopes. Such effector agents may also be useful to increase the half-life of the altered antibodies, e.g., polyethylene glycol.

For use in constructing the antibodies, altered antibodies and fragments of this invention, a non-human species such as bovine, ovine, monkey, chicken, rodent (e.g., murine and rat) may be employed to generate a desirable immunoglobulin upon presentment with human RHAMM receptor or a peptide epitope therefrom. Conventional hybridoma techniques are employed to provide a hybridoma cell line secreting a non-human mAb to the human RHAMM receptor. Such hybridomas are then screened for binding and antagonist activity as described in the Examples section. Alternatively, fully human mAbs can be generated by techniques known to those skilled in the art and used in this invention.

Exemplary antagonist mAbs (monoclonal antibodies) of the present invention are murine mAbs 3E6, 16E10 and 10C5, murine antibodies which can be used for the development of a chimeric or humanized molecule. These mAbs are characterized by antagonist activity on human RHAMM and subsequent inhibition of the ability of RHAMM to bind hyaluronic acid.

The present invention also includes the use of Fab fragments or F(ab′)₂ fragments derived from mAbs directed against the human RHAMM receptor as bivalent fragments. These fragments are useful as agents having antagonist activity at the RHAMM receptor. A Fab fragment contains the entire light chain and amino terminal portion of the heavy chain. An F(ab′)₂ fragment is the fragment formed by two Fab fragments bound by disulfide bonds. The mAbs 3E6, and 16E10 and 10C5 and other similar high affinity antibodies provide sources of Fab fragments and F(ab′)₂ fragments which can be obtained by conventional means, e.g., cleavage of the mAb with the appropriate proteolytic enzymes, papain and/or pepsin, or by recombinant methods. These Fab and F(ab′)₂ fragments are useful themselves as therapeutic, prophylactic or diagnostic agents, and as donors of sequences including the variable regions and CDR sequences useful in the formation of recombinant or humanized antibodies as described herein.

The Fab and F(ab′)₂ fragments can be constructed via a combinatorial phage library (see, e.g., Winter et al., Ann. Rev. Immunol., 12:433-455 (1994)) or via immunoglobulin chain shuffling (see, e.g., Marks et al., Bio/Technology, 10:779-783 (1992)), wherein the Fd or v_{H} immunoglobulin from a selected antibody (e.g., 12H8 or 6A3) is allowed to associate with a repertoire of light chain immunoglobulins, v_{L} (or v_{K}), to form novel Fabs. Conversely, the light chain immunoglobulin from a selected antibody may be allowed to associate with a repertoire of heavy chain immunoglobulins, v_{H} (or Fd), to form novel Fabs. RHAMM receptor antagonist Fabs can be obtained by allowing the Fd of the mABs of the present invention to associate with a repertoire of light chain immunoglobulins. Hence, one is able to recover neutralizing Fabs with unique sequences (nucleotide and amino acid) from the chain shuffling technique.

The mAbs of the present invention may also contribute sequences, such as variable heavy and/or light chain peptide sequences, framework sequences, CDR sequences, functional fragments, and analogs thereof, and the nucleic acid sequences encoding them, useful in designing and obtaining various altered antibodies which are characterized by the antigen binding specificity of the donor antibody.

The nucleic acid sequences of this invention, or fragments thereof, encoding the variable light chain and heavy chain peptide sequences are also useful for mutagenic introduction of specific changes within the nucleic acid sequences encoding the CDRs or framework regions, and for incorporation of the resulting modified or fusion nucleic acid sequence into a plasmid for expression. For example, silent substitutions in the nucleotide sequence of the framework and CDR-encoding regions can be used to create restriction enzyme sites which facilitate insertion of mutagenized CDR and/or framework regions. These CDR-encoding regions can be used in the construction of the humanized antibodies of the invention.

Taking into account the degeneracy of the genetic code, various coding sequences may be constructed which encode the variable heavy and light chain amino acid sequences and CDR sequences of the invention as well as functional fragments and analogs thereof which share the antigen specificity of the donor antibody. The isolated nucleic acid sequences of this invention, or fragments thereof, encoding the variable chain peptide sequences or CDRs can be used to produce altered antibodies, e.g., chimeric or humanized antibodies or other engineered antibodies of this invention when operatively combined with a second immunoglobulin partner.

It should be noted that in addition to isolated nucleic acid sequences encoding portions of the altered antibody and antibodies described herein, other such nucleic acid sequences are encompassed by the present invention, such as those complementary to the native CDR-encoding sequences or complementary to the modified human framework regions surrounding the CDR-encoding regions. Useful DNA sequences include those sequences which hybridize under stringent hybridization conditions to the DNA sequences. See, T. Maniatis et al., Molecular Cloning: A Laboratory Manual, Cold Spring Harbor Laboratory (1982), pp. 387-389. An example of one such stringent hybridization condition is hybridization at 4×SSC at 65° C., followed by a washing in 0.1×SSC at 65° C. for one hour. Alternatively, an exemplary stringent hybridization condition is 50% formamide, 4×SSC at 42° C. Preferably, these hybridizing DNA sequences are at least about 18 nucleotides in length, i.e., about the size of a CDR.

Altered immunoglobulin molecules can encode altered antibodies which include engineered antibodies such as chimeric antibodies and humanized antibodies. A desired altered immunoglobulin coding region contains CDR-encoding regions that encode peptides having the antigen specificity of a RHAMM receptor antibody, preferably a high-affinity antagonist antibody such as provided by the present invention, inserted into a first immunoglobulin partner such as a human framework or human immunoglobulin variable region.

Preferably, the first immunoglobulin partner is operatively linked to a second immunoglobulin partner. The second immunoglobulin partner is defined above, and may include a sequence encoding a second antibody region of interest, for example an Fc region. Second immunoglobulin partners may also include sequences encoding another immunoglobulin to which the light or heavy chain constant region is fused in frame or by means of a linker sequence. Engineered antibodies directed against functional fragments or analogs of the RHAMM receptor may be designed to elicit enhanced binding with the same antibody.

The second immunoglobulin partner may also be associated with effector agents as defined above, including non-protein carrier molecules, to which the second immunoglobulin partner may be operatively linked by conventional means.

Fusion or linkage between the second immunoglobulin partners, e.g., antibody sequences, and the effector agent may be by any suitable means, e.g., by conventional covalent or ionic bonds, protein fusions, or hetero-bifunctional cross-linkers, e.g., carbodiimide, glutaraldehyde and the like. Such techniques are known in the art and are described in conventional chemistry and biochemistry texts.

Additionally, conventional linker sequences which simply provide for a desired amount of space between the second immunoglobulin partner and the effector agent may also be constructed into the altered immunoglobulin coding region. The design of such linkers is well known to those of skill in the art.

In addition, signal sequences for the molecules of the invention may be modified by techniques known to those skilled in the art to enhance expression.

A preferred altered antibody contains a variable heavy and/or light chain peptide or protein sequence having the antigen specificity of mAb 3E6, 16E 10 or 10C5, e.g., the V_{H} and V_{L} chains. Still another desirable altered antibody of this invention is characterized by the amino acid sequence containing at least one, and preferably all of the CDRs of the variable region of the heavy and/or light chains of the murine antibody molecule 3E6, 16E10 or 10C5 with the remaining sequences being derived from a human source, or a functional fragment or analog thereof.

In a further embodiment, the altered antibody of the invention may have attached to it an additional agent. For example, recombinant DNA technology may be used to produce an altered antibody of the invention in which the Fc fragment or CH2 CH3 domain of a complete antibody molecule has been replaced by an enzyme or other detectable molecule, i.e., a polypeptide effector or reporter molecule. Other additional agents include toxins, antiproliferative drugs and radionuclides.

The second immunoglobulin partner may also be operatively linked to a non-immunoglobulin peptide, protein or fragment thereof heterologous to the CDR-containing sequence having antigen specificity to the RHAMM receptor. The resulting protein may exhibit both antigen specificity and characteristics of the non-immunoglobulin upon expression. That fusion partner characteristic may be, e.g., a functional characteristic such as another binding or receptor domain or a therapeutic characteristic if the fusion partner is itself a therapeutic protein or additional antigenic characteristics.

Another desirable protein of this invention may comprise a complete antibody molecule, having full length heavy and light chains or any discrete fragment thereof, such as the Fab or F(ab′)₂ fragments, a heavy chain dimer or any minimal recombinant fragments thereof such as an Fᵥ or a single-chain antibody (SCA) or any other molecule with the same specificity as the selected donor mAb, e.g., the 10C5, 16E10 or 3E6 mAB. Such protein may be used in the form of an altered antibody or may be used in its unfused form.

Whenever the second immunoglobulin partner is derived from an antibody different from the donor antibody, e.g., any isotype or class of immunoglobulin framework or constant regions, an engineered antibody results. Engineered antibodies can comprise immunoglobulin constant regions and variable framework regions from one source, e.g., the acceptor antibody, and one or more (preferably all) CDRs from the donor antibody. In addition, alterations, e.g., deletions, substitutions, or additions, of the acceptor mAb light and/or heavy variable domain framework region at the nucleic acid or amino acid levels, or the donor CDR regions may be made in order to retain donor antibody antigen binding specificity.

Such engineered antibodies are designed to employ one (or both) of the variable heavy and/or light chains of the RHAMM receptor mAb (optionally modified as described) or one or more of the heavy or light chain CDRs. The engineered antibodies of the invention exhibit antagonist activity.

Such engineered antibodies may include a humanized antibody containing the framework regions of a selected human immunoglobulin or subtype or a chimeric antibody containing the human heavy and light chain constant regions fused to the RHAMM receptor mAb functional fragments. A suitable human (or other animal) acceptor antibody may be one selected from a conventional database, e.g., the KABAT® database, Los Alamos database, and Swiss Protein database, by homology to the nucleotide and amino acid sequences of the donor antibody. A human antibody characterized by a homology to the V region frameworks of the donor antibody or V region subfamily consensus sequences (on an amino acid basis) may be suitable to provide a heavy chain variable framework region for insertion of the donor CDRs. A suitable acceptor antibody capable of donating light chain variable framework regions may be selected in a similar manner. It should be noted that the acceptor antibody heavy and light chains are not required to originate from the same acceptor antibody.

Preferably, the heterologous framework and constant regions are selected from human immunoglobulin classes and isotypes, such as IgG (subtypes 1 through 4), IgM, IgA, and IgE. IgG1, k and IgG4, k are preferred. Particularly preferred is IgG 4, k. Most particularly preferred is the IgG4 subtype variant containing the mutations S228P and L235E (PE mutation) in the heavy chain constant region which results in reduced effector function. This IgG4 subtype variant is known herein as IgG4PE. See U.S. Pat. Nos. 5,624,821 and 5,648,260.

The acceptor antibody need not comprise only human immunoglobulin protein sequences. For instance, a gene may be constructed in which a DNA sequence encoding part of a human immunoglobulin chain is fused to a DNA sequence encoding a non-immunoglobulin amino acid sequence such as a polypeptide effector or reporter molecule.

A particularly preferred humanized antibody contains CDRs of 3E6, 16E10 or 10C5 mAb inserted onto the framework regions of a selected human antibody sequence. For antagonist humanized antibodies, one, two or preferably three CDRs from the antibody heavy chain and/or light chain variable regions are inserted into the framework regions of the selected human antibody sequence, replacing the native CDRs of the human antibody.

Preferably, in a humanized antibody, the variable domains in both human heavy and light chains have been engineered by one or more CDR replacements. It is possible to use all six CDRs, or various combinations of less than the six CDRs. Preferably all six CDRs are replaced. It is possible to replace the CDRs only in the human heavy chain, using as light chain the unmodified light chain from the human acceptor antibody. Still alternatively, a compatible light chain may be selected from another human antibody by recourse to conventional antibody databases. The remainder of the engineered antibody may be derived from any suitable acceptor human immunoglobulin.

The engineered humanized antibody thus preferably has the structure of a natural human antibody or a fragment thereof, and possesses the combination of properties required for effective therapeutic use, e.g., treatment of angiogenic diseases such as diabetic retinopathy and macular degeneration or treatment of proliferative diseases, such as cancer, arthritis, psoriasis and atherosclerosis.

It will be understood by those skilled in the art that an engineered antibody may be further modified by changes in variable domain amino acids without necessarily affecting the specificity and high affinity of the donor antibody (i.e., an analog). It is anticipated that heavy and light chain amino acids may be substituted by other amino acids either in the variable domain frameworks or CDRs or both. These substitutions could be supplied by the donor antibody or consensus sequences from a particular subgroup.

In addition, the constant region may be altered to enhance or decrease selective properties of the molecules of this invention. For example, dimerization, binding to Fc receptors, or the ability to bind and activate complement (see, e.g., Angal et al., Mol. Immunol, 30, 105-108 (1993), Xu et al., J. Biol. Chem, 269, 3469-3474 (1994), Winter et al., EP 307434-B).

An altered antibody which is a chimeric antibody differs from the humanized antibodies described above by providing the entire non-human donor antibody heavy chain and light chain variable regions, including framework regions, in association with human immunoglobulin constant regions for both chains. It is anticipated that chimeric antibodies which retain additional non-human sequence relative to humanized antibodies of this invention may elicit a significant erythropoietic response in humans. Such antibodies are useful in the prevention of and for treating of proliferative diseases.

Preferably, the variable light and/or heavy chain sequences and the CDRs of the mAbs of the invention or other suitable donor mAbs and their encoding nucleic acid sequences, are utilized in the construction of altered antibodies, preferably humanized antibodies, of this invention, by the following process. The same or similar techniques may also be employed to generate other embodiments of this invention.

A hybridoma producing a selected donor mAb, e.g., one of the murine antibodies of the invention, is conventionally cloned and the DNA of its heavy and light chain variable regions obtained by techniques known to one of skill in the art, e.g., the techniques described in Sambrook et al., Molecular Cloning: A Laboratory Manual, 2nd edition, Cold Spring Harbor Laboratory (1989). The variable heavy and light regions containing at least the CDR-encoding regions and those portions of the acceptor mAb light and/or heavy variable domain framework regions required in order to retain donor mAb binding specificity, as well as the remaining immunoglobulin-derived parts of the antibody chain derived from a human immunoglobulin, are obtained using polynucleotide primers and reverse transcriptase. The CDR-encoding regions are identified using a known database and by comparison to other antibodies.

A mouse/human chimeric antibody may then be prepared and assayed for binding ability. Such a chimeric antibody contains the entire non-human donor antibody V_{H} and V_{L} regions, in association with human Ig constant regions for both chains.

Homologous framework regions of a heavy chain variable region from a human antibody are identified using computerized databases, e.g., KABAT®, and a human antibody characterized by a homology to the V region frameworks of the donor antibody or V region subfamily consensus sequences (on an amino acid basis) to one of the antibodies of the invention is selected as the acceptor antibody. The sequences of synthetic heavy chain variable regions containing the CDR-encoding regions within the human antibody frameworks are designed with optional nucleotide replacements in the framework regions to incorporate restriction sites. This designed sequence is then synthesized using long synthetic oligomers. Alternatively, the designed sequence can be synthesized by overlapping oligonucleotides, amplified by polymerase chain reaction (PCR), and corrected for errors. A suitable light chain variable framework region can be designed in a similar manner.

A humanized antibody may be derived from the chimeric antibody, or preferably, made synthetically by inserting the donor mAb CDR-encoding regions from the heavy and light chains appropriately within the selected heavy and light chain framework. Alternatively, a humanized antibody of the invention may be prepared using standard mutagenesis techniques. Thus, the resulting humanized antibody contains human framework regions and donor mAb CDR-encoding regions. There may be subsequent manipulation of framework residues. The resulting humanized antibody can be expressed in recombinant host cells, e.g., COS, CHO or myeloma cells.

A conventional expression vector or recombinant plasmid is produced by placing these coding sequences for the altered antibody in operative association with conventional regulatory control sequences capable of controlling the replication and expression in, and/or secretion from, a host cell. Regulatory sequences include promoter sequences, e.g., CMV or Rous Sarcoma virus promoter, and signal sequences, which can be derived from other known antibodies. Similarly, a second expression vector can be produced having a DNA sequence which encodes a complementary antibody light or heavy chain. Preferably, this second expression vector is identical to the first except with respect to the coding sequences and selectable markers, in order to ensure, as much as possible, that each polypeptide chain is functionally expressed. Alternatively, the heavy and light chain coding sequences for the altered antibody may reside on a single vector.

A selected host cell is co-transfected by conventional techniques with both the first and second vectors (or simply transfected by a single vector) to create the transfected host cell of the invention comprising both the recombinant or synthetic light and heavy chains. The transfected cell is then cultured by conventional techniques to produce the engineered antibody of the invention. The humanized antibody which includes the association of both the recombinant heavy chain and/or light chain is screened from culture by an appropriate assay such as ELISA or RIA. Similar conventional techniques may be employed to construct other altered antibodies and molecules of this invention.

Suitable vectors for the cloning and subcloning steps employed in the methods and construction of the compositions of this invention may be selected by one of skill in the art. For example, the pUC series of cloning vectors, such as pUC19, which is commercially available from supply houses, such as Amersham or Pharmacia, may be used. Additionally, any vector which is capable of replicating readily, has an abundance of cloning sites and selectable genes (e.g., antibiotic resistance) and is easily manipulated may be used for cloning. Thus, the selection of the cloning vector is not a limiting factor in this invention.

Similarly, the vectors employed for expression of the engineered antibodies according to this invention may be selected by one of skill in the art from any conventional vector. The vectors also contain selected regulatory sequences (such as CMV or Rous Sarcoma virus promoters) which direct the replication and expression of heterologous DNA sequences in selected host cells. These vectors contain the above-described DNA sequences which code for the engineered antibody or altered immunoglobulin coding region. In addition, the vectors may incorporate the selected immunoglobulin sequences modified by the insertion of desirable restriction sites for ready manipulation.

The expression vectors may also be characterized by genes suitable for amplifying expression of the heterologous DNA sequences, e.g., the mammalian dihydrofolate reductase gene (DHFR). Other preferable vector sequences include a poly A signal sequence, such as from bovine growth hormone (BGH) and the betaglobin promoter sequence (betaglopro). The expression vectors useful herein may be synthesized by techniques well known to those skilled in this art.

The components of such vectors, e.g., replicons, selection genes, enhancers, promoters, signal sequences and the like, may be obtained from commercial or natural sources or synthesized by known procedures for use in directing the expression and/or secretion of the product of the recombinant DNA in a selected host. Other appropriate expression vectors of which numerous types are known in the art for mammalian, bacterial, insect, yeast and fungal expression may also be selected for this purpose.

The present invention also provides a cell line transfected with a recombinant plasmid containing the coding sequences of the engineered antibodies or altered immunoglobulin molecules thereof. Host cells useful for the cloning and other manipulations of these cloning vectors are also conventional. However, most desirably, cells from various strains of E. coli are used for replication of the cloning vectors and other steps in the construction of altered antibodies of this invention.

Suitable host cells or cell lines for the expression of the engineered antibody or altered antibody of the invention are preferably mammalian cells such as CHO, COS, a fibroblast cell (e.g., 3T3) and myeloid cells, and more preferably a CHO or a myeloid cell. Human cells may be used, thus enabling the molecule to be modified with human glycosylation patterns. Alternatively, other eukaryotic cell lines may be employed. The selection of suitable mammalian host cells and methods for transformation, culture, amplification, screening and product production and purification are known in the art. See, e.g., Sambrook et al., supra.

Bacterial cells may prove useful as host cells suitable for the expression of the recombinant Fabs of the present invention (see, e.g., Plückthun, A., Immunol. Rev., 130, 151-188 (1992)). However, due to the tendency of proteins expressed in bacterial cells to be in an unfolded or improperly folded form or in a non-glycosylated form, any recombinant Fab produced in a bacterial cell would have to be screened for retention of antigen binding ability. If the molecule expressed by the bacterial cell was produced in a properly folded form, that bacterial cell would be a desirable host. For example, various strains of E. coli used for expression are well-known as host cells in the field of biotechnology. Various strains of B. subtilis, Streptomyces, other bacilli and the like may also be employed.

Where desired, strains of yeast cells known to those skilled in the art are also available as host cells, as well as insect cells, e.g. Drosophila and Lepidoptera, and viral expression systems. See, e.g. Miller et al., Genetic Engineering, 8, 277-298, Plenum Press (1986) and references cited therein.

The general methods by which the vectors of the invention may be constructed, the transfection methods required to produce the host cells of the invention, and culture methods necessary to produce the altered antibody of the invention from such host cell are all conventional techniques. Likewise, once produced, the altered antibodies of the invention may be purified from the cell culture contents according to standard procedures of the art, including ammonium sulfate precipitation, affinity columns, column chromatography, gel electrophoresis and the like. Such techniques are within the skill of the art and do not limit this invention.

Yet another method of expression of the humanized antibodies may utilize expression in a transgenic animal, such as described in U.S. Pat. No. 4,873,316. This relates to an expression system using the animal's casein promoter which when transgenically incorporated into a mammal permits the female to produce the desired recombinant protein in its milk.

Once expressed by the desired method, the engineered antibody is then examined for in vitro activity by use of an appropriate assay. Additionally, in vitro assays such as the Matrigel vascularization model may also be used to determine antagonist activity prior to subsequent human clinical studies performed to evaluate the persistence of the engineered antibody in the body despite the usual clearance mechanisms.

Following the procedures described for humanized antibodies prepared from the antibodies of the invention, one of skill in the art may also construct humanized antibodies from other donor antibodies, variable region sequences and CDR peptides described herein. Engineered antibodies can be produced with variable region frameworks potentially recognized as “self” by recipients of the engineered antibody. Modifications to the variable region frameworks can be implemented to effect increases in antigen binding and antagonist activity without appreciable increased immunogenicity for the recipient.

This invention also relates to a method for treating or preventing proliferative disease states in a mammal comprising administering to a subject in need thereof an effective dose of a RHAMM receptor antagonist mAb of the invention. The mAb can include one or more of the engineered antibodies or altered antibodies described herein or fragments thereof.

Proliferative disease states include leukemia, solid tumor cancer, lymphoma, and cancer of soft tissue, brain, esophagus, stomach, pancreas, liver, lung, bladder, bone, prostate, ovary, cervix, skin, breast, testicular, kidney, head, neck and colon; chronic inflammatory proliferative diseases selected from the group consisting of psoriasis, inflammatory bowel disease and rheumatoid arthritis; proliferative cardiovascular diseases; proliferative ocular diseases and benign hyperproliferative diseases.

The altered antibodies, antibodies and fragments thereof of this invention may also be used in conjunction with other antibodies, particularly human mAbs reactive with other markers (epitopes) responsible for the condition against which the engineered antibody of the invention is directed.

The RHAMM receptor antagonist antibodies of the invention can be formulated into pharmaceutical compositions and administered in the same manner as described for mature proteins. See, e.g., International Patent Application, Publication No. WO90/02762 (Mar. 22 1990). Generally, these compositions contain a therapeutically effective amount of an antagonist antibody of this invention and an acceptable pharmaceutical carrier. Suitable carriers are well known to those of skill in the art and include, for example, saline. Alternatively, such compositions may include conventional delivery systems into which protein of the invention is incorporated. Optionally, these compositions may contain other active ingredients.

The therapeutic agents of this invention may be administered by any appropriate internal route, and may be repeated as needed, e.g., as frequently as one to three times daily for between 1 day to about three weeks to once per week or once biweekly. Preferably, the antagonist antibody is administered less frequently than is the ligand, when it is used therapeutically. The dose and duration of treatment relates to the relative duration of the molecules of the present invention in the human circulation, and can be adjusted by one of skill in the art depending upon the condition being treated and the general health of the patient.

As used herein, the term “pharmaceutical” includes veterinary applications of the invention. The term “therapeutically effective amount” refers to that amount of a receptor antagonist antibody, which is useful for alleviating a selected condition. These therapeutic compositions of the invention may be administered to mimic the effect of the normal receptor ligand.

The mode of administration of the therapeutic agent of the invention may be any suitable route which delivers the agent to the host. The altered antibodies, antibodies, engineered antibodies, and fragments thereof, and pharmaceutical compositions of the invention are particularly useful for parenteral administration, i.e., subcutaneously, intramuscularly, intravenously or intranasally.

Therapeutic agents of the invention may be prepared as pharmaceutical compositions containing an effective amount of the engineered (e.g., humanized) antibody of the invention as an active ingredient in a pharmaceutically acceptable carrier. In the compositions of the invention, an aqueous suspension or solution containing the engineered antibody, preferably buffered at physiological pH, in a form ready for injection is preferred. The compositions for parenteral administration will commonly comprise a solution of the engineered antibody of the invention or a cocktail thereof dissolved in an pharmaceutically acceptable carrier, preferably an aqueous carrier. A variety of aqueous carriers may be employed, e.g., 0.4% saline, 0.3% glycine and the like. These solutions are sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, etc. The concentration of the antibody of the invention in such pharmaceutical formulation can vary widely, i.e., from less than about 0.5%, usually at or at least about 1% to as much as 15 or 20% by weight and will be selected primarily based on fluid volumes, viscosities, etc., according to the particular mode of administration selected.

Thus, a pharmaceutical composition of the invention for intramuscular injection could be prepared to contain 1 mL sterile buffered water, and between about 1 ng to about 100 mg, e.g. about 50 ng to about 30 mg or more preferably, about 5 mg to about 25 mg, of an engineered antibody of the invention. Similarly, a pharmaceutical composition of the invention for intravenous infusion could be made up to contain about 250 ml of sterile Ringer's solution, and about 1 mg to about 30 mg and preferably 5 mg to about 25 mg of an engineered antibody of the invention. Actual methods for preparing parentally administrable compositions are well known or will be apparent to those skilled in the art and are described in more detail in, for example, “Remington's Pharmaceutical Science”, 15th ed., Mack Publishing Company, Easton, Pa.

It is preferred that the therapeutic agent of the invention, when in a pharmaceutical preparation, be present in unit dose forms. The appropriate therapeutically effective dose can be determined readily by those of skill in the art. To effectively treat anemia in a human or other animal, one dose of approximately 0.01 mg to approximately 20 mg per kg body weight of a protein or an antibody of this invention should be administered parentally, preferably i.v. or i.m. Such dose may, if necessary, be repeated at appropriate time intervals selected as appropriate by a physician during the response period.

The present invention will now be described with reference to the following specific, non-limiting examples.

### EXAMPLE 1

### Preparation of the Antigen

The human RHAMM cDNA (Wang et al, 1996, Gene vol174:299-306) expression construct was constructed which has the glutathione-S-transferase gene fused to the amino terminus of RHAMM via a linker containing a thrombin cleavage site. This construct was cloned into the Baculovirus expression vector, pFASTBAC, and this was used to make the viral stock for the subsequent infection. Spodoptera frugiperda cells (Sf9) were infected with the virus expressing the GST-RHAMM and the cells were grown for 3 days, then harvested and frozen down.

The GST-RHAMM protein was purified from Sf9 cell pellets by lysis via sonication and chromatography over Glutathione Sepharose 4B. The GST-RHAMM was eluted from the column with 10 mM Glutathione. In some cases, the RHAMM protein was cleaved from the beads by thrombin to isolate RHAMM with the GST tag removed.

### Preparation and Screening of RHAMM Antagonist Monoclonal Antibodies

3-Mice (F1 hybrids of Balb/c and C57BL/6) were immunized subcutaneously with recombinant human RHAMM protein in RIBI adjuvant and boosted with the same. A splenectomy was performed 3-4 days following the final immunization. Mouse spleen cells were used to prepare hybridomas by standard procedures, (Zola, H. Ed., Monoclonal Antibodies, CRC Press Inc. (1987)). Positive hybridomas were cloned by the limiting dilution method, generating mAbs 3E6, 10C5 and 16E10).

### Immunoassay

To determine the specificity of the anti-RHAMM mAbs generated, 96-well plates were coated with GST-RHAMM or RHAMM in which the GST tag had been enzymatically removed. The wells were then blocked with BSA. All the following incubations were performed in a shaker-incubator at RT. After washing the wells, assay buffer and mAb/hybridoma supernatants were added and incubated for 60 min. After washing the wells, biotinylated anti-mouse antibody in assay buffer was added for 45 min, the wells washed and Eu³⁺ labeled streptavidin in assay buffer was added for 30 min, the wells were washed, then enhancer (Wallac) was added and incubated for 5 min at RT and the fluorescence measured. All positive hybridomas showed binding to GST-RHAMM.

### HA Binding Assay

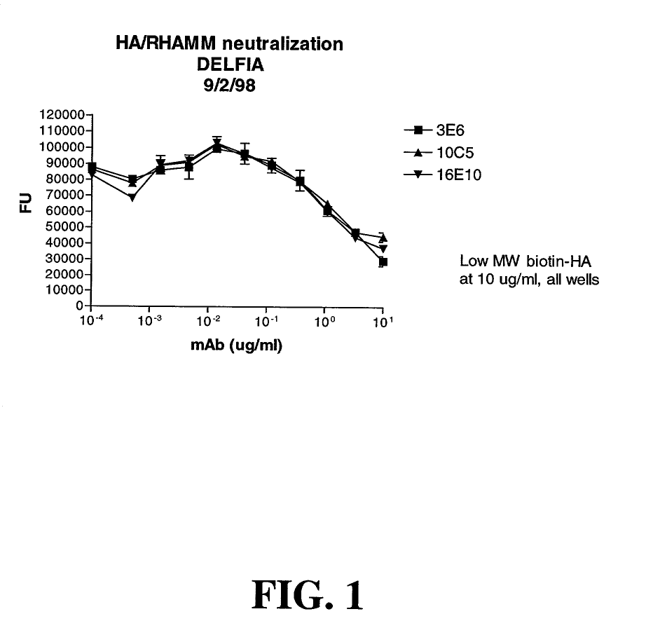

To measure the ability of RHAMM mAbs to inhibit the binding of HA to RHAMM, an HA binding assay was used. 96-well plates were coated with GST-RHAMM or RHAMM in which the GST tag had been enzymatically removed. The wells were then blocked with BSA. All the following incubations were performed 37° C. After washing the wells, assay buffer and mAb/hybridoma supernatants were added, followed by biotinylated HA. Plates were incubated for 60 min. After washing the wells, Eu³⁺ labeled streptavidin in assay buffer was added for 30 min, the wells were washed, then enhancer (Wallac) was added and incubated for 5 min at RT and the fluorescence measured. In the absence of RHAMM mAbs, HA bound the RHAMM in a reproducible concentration dependent manner. Monoclonal antibodies 3E6, 10C5, and 16E10 were able to inhibit the binding of HA to RHAMM (FIG. 1)

### Purification of Mabs

Monoclonal antibodies 3E6, 10C5, and 16E10 were purified by protein-A chromatography per the manufacturer's instructions from the selected hybridoma supernatants. Mabs were >95% pure by SDS-PAGE.

### Affinity Measurements of Monoclonal Antibodies

The affinity of the purified mAbs was measured in the BIAcore. Using a flow rate of 10 ul/min, the mAb (diluted in HBS buffer) was injected over a rabbit anti-mouse IgG Fc surface, followed by buffer flow and the RU recorded. RHAMM diluted in HBS buffer was then injected for 180s followed by buffer flow for 150s and regeneration of the sensor chip surface with an injection of 0.1 M phosphoric acid. BIAcore software was used for association and dissociation-phase analysis. The murine monoclonal antibodies bound to soluble monomeric RHAMM. The on-rates (kₐₛₛ) and off-rates (k_{diss}) were calculated. Together, these yield a calculated equilibrium constant (K_{D}) of 1.8×10⁻¹⁰ M for mAb 3E6; 4.7×10⁻¹⁰ M for mAb 16E10 and 0.9×10⁻⁹ M for mAb 10C5.

### Epitope Analysis of Monoclonal Antibodies

The epitope analysis of the purified mAbs was measured in the BIAcore. Using a flow rate of 10 ul/min, the first mAb (diluted in HBS buffer) was injected over a rabbit anti-mouse IgG Fc surface, followed by, an injection of RHAMM for 240s, an injection of blocking mAbs for 48s and an injection of the second mAb for 240s. The surface was regenerated by an injection of 0.1M phosphoric acid and the RU was recorded after each injection. It was found that mAbs 3E6 and 10C5 have different epitopes, mAbs 16E10 and 10C5 have different epitopes and that mAbs 16E10 and 3E6 have similar or overlapping epitopes.

### EXAMPLE 2

### Functional Screening of RHAMM Antagonist Monoclonal Antibodies MAP Kinase Activation Assay

This assay measures the activation of p42/44 MAP kinase via the ras pathway by PDGF treatment, and can be used to test whether RHAMM antibodies can block the activation. Cell types used are normal human lung fibroblasts (NHLF) or normal human dermal fibroblasts (NHDF) (Clonetics, San Diego, Calif.). Cells are plated at low density and serum starved for 72 hours. After 72 hours of serum starvation the cells should be ˜50% confluent. Cells are treated with the monoclonal antibodies at concentrations between 1 and 50 ug/ml for 45 min, followed by stimulation with 2 ng/ml platelet derived growth factor (PDGF) for 15 min. The cells are then lysed in RIPA buffer containing sodium orthovanadate. Activation of p42/44 MAP kinase is assayed by western blotting using an antibody specific for the activated phosphorylated form of p42/44 (New England Biolabs, Boston, Mass.). The data (not shown) demonstrated that RHAMM monoclonal antibodies 10C5, 16E10 and 3E6 were able to block the PDGF-induced activation of p42/44 MAP kinase, indicating that these antibodies are interfering with ras-mediated signaling.

### EXAMPLE 3

### Cloning and Sequencing of 16E10 Light and Heavy Chain cDNAs

The amino acid sequences of 12 light chain amino-terminal residues and 13 heavy chain amino-terminal residues of 16E10 were determined. The amino terminus of the heavy chain was blocked with pyroglutamic acid. It was successfully deblocked enzymatically using pyroglutamate aminopeptidase.

Total 16E10 RNA was purified, reverse transcribed and PCR amplified. For the heavy chain, the RNA/DNA hybrid was PCR amplified using a mouse IgG CH1-specific primer and a degenerate primer based on the N-term protein sequence. Similarly, for the light chain, the RNA/DNA hybrid was PCR amplified using a mouse C kappa primer and a degenerate primer based on the N-term protein sequence. PCR products of the appropriate size, i.e., ˜350 bp, were cloned into a plasmid vector, and sequenced by a modification of the Sanger method. In each case the sequence of VH and Vk clones were compared to generate a consensus 16E10 heavy chain variable region sequence (SEQ ID NOs: 1 and 2) and consensus 16E10 light chain variable region sequence (SEQ ID NOs: 3 and 4), respectively. The heavy chain CDR 1, 2 and 3 amino acid sequences are shown in SEQ ID NOs: 5, 6 and 7, respectively. The light chain CDR 1, 2 and 3 amino acid sequences are shown in SEQ ID NOs: 8, 9 and 10, respectively.

### Deposited Materials

A deposit containing the monoclonal antibodies of the invention designated ATCC 10C5, 16E10 and 3E6 has been deposited with the American Type Culture Collection at 10801 University Blvd., Manassas, Va. 20110-2209, Telephone 703-375-2700.

The deposit of the deposited mABs has been made under the terms of the Budapest Treaty on the International Recognition of the Deposit of Micro-organisms for Purposes of Patent Procedure. The deposited mABs are provided merely as convenience to those of skill in the art and is not an admission that a deposit is required for enablement, such as that required under 35 U.S.C. § 112. A license may be required to make, use or sell the deposited strain, and compounds derived therefrom, and no such license is hereby granted.

The present invention may be embodied in other specific forms without departing from the spirit or essential attributes thereof, and, accordingly, reference should be made to the appended claims, rather than to the foregoing specification, as indicating the scope of the invention.

We claim:

1. A RHAMM (receptor for hyaluronic acid mediated motility) receptor antagonist antibody having the identifying characteristics of monoclonal antibody 10C5, 16E10 or 3E6.

2. The antibody of claim 1 which is monoclonal antibody 10C5.

3. The antibody of claim 1 which is monoclonal antibody 16E10.

4. The antibody of claim 1 which is monoclonal antibody 3E6.

5. A polypeptide comprising an immunoglobulin complementarily determining region of the antibody of claim 2.

6. A polypeptide comprising an immunoglobulin complementarily determining region of the antibody of claim 3.

7. A polypeptide comprising an immunoglobulin complementarily determining region of the antibody of claim 4.

8. An isolated polynucleotide encoding a polypeptide of claim 5.

9. An isolated polynucleotide encoding a polypeptide of claim 6.

10. An isolated polynucleotide encoding a polypeptide of claim 7.

11. A method for treating or preventing proliferative disease states in a mammal comprising administering an effective dose of a RHAMM receptor antibody.

12. The method of claim 11 wherein the proliferative disease states comprise malignant cancers selected from the group consisting of leukemia, solid tumor cancer, lymphoma, and cancer of soft tissue, brain, esophagus, stomach, pancreas, liver, lung, bladder, bone, prostate, ovary, cervix, skin, breast, testicular, kidney, head, neck and colon.

13. The method of claim 11 wherein the proliferative disease states comprise the chronic inflammatory proliferative diseases psoriasis, inflammatory bowel disease or rheumatoid arthritis.

14. The method of claim 11 wherein the proliferative disease states comprise proliferative cardiovascular diseases, proliferative ocular diseases or benign hyperproliferative diseases.

15. A pharmaceutical composition comprising the monoclonal antibody of claim 1.

16. A pharmaceutical composition comprising monoclonal antibody 10C5, 16E10 or 3E6.

17. A pharmaceutical composition comprising a polypeptide comprising an immunoglobulin complementarily determining region of monoclonal antibody 10C5, 16E10 or 3E6.

18. A hybridoma cell line having the identifying characteristics of a cell line which produces monoclonal antibody 3E6, 10C5 or 16E10.

19. A monoclonal antibody identified as ATCC 10C5, ATCC 16E10 or ATCC 3E6.

20. An antibody comprising a heavy chain variable region (V_{H}) polypeptide as set forth in SEQ ID NO: 2 and a light chain variable region (V_{L}) polypeptide as set forth in SEQ ID NO: 4.

21. An isolated polynucleotide encoding the polypeptide of SEQ ID NO: 2 or SEQ ID NO: 4.

22. An immunoglobulin heavy chain complementarity determining region (CDR), the amino acid sequence of which is shown in SEQ ID NOs: 5, 6 or 7.

23. An isolated polynucleotide encoding the CDR of claim 22.

24. An immunoglobulin light chain CDR, the amino acid sequence of which is shown in SEQ ID NOs: 8, 9 or 10.

25. An isolated polynucleotide encoding the CDR of claim 24.
